(19) United States
(12) Patent Application Publication
(10) Pub. No.: US 2007/0127789 A1
(43) Pub. Date: Jun. 7, 2007

(54) Method for three dimensional multi-phase quantitative tissue evaluation

(76) Inventors: Bernice E. Hoppel, Delafield, WI (US); Gopal B. Avinash, New Berlin, WI (US); Kelly Lynn Piacsek, Pewaukee, WI (US)

Correspondence Address: CANTOR COLBURN, LLP, 55 GRIFFIN ROAD SOUTH, BLOOMFIELD, CT, 06002, US

(21) Appl. No.: 11/272,118

(22) Filed: Nov. 10, 2005

(51) Int. Cl. G06K 9/00; A61B 5/05

(52) U.S. Cl. 382/128; 600/407

(57) ABSTRACT

A method of evaluating tissue of an organ includes accessing image data and processing the image data to quantify at least one feature of interest in the tissue. The image data is derived from a computed tomography acquisition system and includes multiple phases of acquired whole organ data. Each phase is acquired within five gantry rotations of the acquisition system using an acquisition protocol.

### BACKGROUND OF THE INVENTION

This invention relates generally to imaging systems and methods of using thereof, specifically to a method of imaging organ systems with a perfusion and/or a viability protocol.

The diagnosis of myocardial tissue viability after a heart attack focuses on whether there will be functional improvement of dysfunctional myocardium after revascularization therapy. Both single photon emission computed tomography (SPECT) and contrast enhanced magnetic resonance imaging (MRI) have been able to measure the viability of tissue. SPECT is a relatively inexpensive method for functionally measuring the viability within the myocardium. In comparison, MRI is a better standard of measurement because of the spatial resolution that is available for definition of infarcts, and in particular, non-transmural infarcts.

MRI uses both anatomical and functional methods to determine the viability of the myocardium. MRI also provides functional information about flow through microvasculature within the myocardium by continuous imaging (scanning) following the injection of a contrast agent. This allows for the visualization of the perfusion or flow through regions of myocardium that maybe affected. Regions lacking microvasculature flow show up as hypo-enhancement due to the lack of contrast agent flowing through that area. Additionally, a technique called delayed hyper-enhancement MRI is employed to reveal the extent of injured myocardium in dysfunctional myocardial tissue. Injured myocardium may recover contractile function once blood flow delivering oxygen and substrates is restored, either spontaneously or following revascularization.

In delayed hyper-enhancement, a contrast agent is infused either continuously or as a bolus via an intravenous route and an image is taken 10-15 minutes following infusion. In normal myocardium, the infused contrast agent is excluded from intracellular compartments. However, in injured myocardium, the sarcolemmal membrane of myocytes become permeable allowing contrast agent to accumulate, which results in the observed hyper-enhancement. Thus, lack of contractile function (hypokinesia) and absence of hyper-enhancement (preserved integrity of the sarcolemmal membrane of myocytes) may indicate the presence of hibernating, or viable, myocardium, which may improve after revascularization of the artery supplying a particular organ or anatomical part. Magnetic resonance (MR) viability imaging using the above, described combination of anatomical and functional methods can reliably differentiate areas of hibernating (viable) from infracted (non-viable) myocardium following a heart attack.

MR viability studies have also been able to report changes in right and left ventricular volume, changes in myocardial thickness, as well as signal intensity changes from phase contrast, perfusion and delayed enhancement studies. From these MR viability studies, one can calculate stroke volume, ejection fraction, percent wall thickness of non-viable tissue and blood flow from the larger vessels. Additionally, perfusion data from MR can calculate relative blood flow, blood volume and mean transit time. Essentially, MRI produces qualitative information concerning perfusion defects and myocardial viability.

With the advent of high-speed volumetric scanners, computed tomography (CT) can be used to differentiate between viable and non-viable myocardium, peri- and post-ischemic attack, in much the same way as MR imaging but with the additional capability of quantitative information concerning perfusion defects and myocardial viability.

One protocol for CT consists of an initial high-resolution, electrocardiogram (ECG) gated, non-contrast enhanced helical scanning of the whole heart with a breath-hold. This step acquires a high quality volume image of the myocardium at a baseline. The second step is another ECG gated, helical scanning of the whole heart with breath-hold but during intravenous injection of a contrast agent, which acquires computed tomography angiography (CTA) images to document stenoses and atherosclerotic plaques of coronary vessels. The same CTA study can be reformatted to allow investigation of wall motion and wall thickening. The third step is a CT perfusion scan of the heart with another contrast injection. The contrast agent is followed in its “first pass” through the myocardium where perfusion or flow defects can be observed. At some time delay after the perfusion scan and contrast injection, a final high-resolution, ECG gated, helical scanning of the whole heart with breath-hold image is acquired. Subtraction of the baseline helical scan from this last helical scan will highlight hyper-enhanced and hence injured myocardium. Furthermore, CT can accurately quantify the effect from the contrast agent because the image intensity is proportional to attenuation of the x-rays. Therefore, accurate measurements of blood flow, blood volume, extraction fraction and permeability can be obtained.

Within CT-based research, perfusion and viability imaging is limited to small volumes of imaging. Small volume imaging is not able to assess the extent of cardiac injury to the entire organ and, therefore, may be inadequate for routine clinical use.

Multi-detector CT systems, particularly the new volume computed tomography (VCT) scanners, whole organs can be imaged with essentially four-dimensional computed tomography (4DCT) quantitatively. The multi-phasic quantitative three-dimensional (3D) CT imaging allows for complete coverage of the organ, such as a heart, for a more realistic assessment of the injury caused by ischemic attack and prognosis for treatment in a quantitative manner.

Multiple 3D image data sets, collected with the protocol described above, require extensive analysis and comparison between the wall motion, coronary CTA images, perfusion and delayed hyper-enhancement data sets. Visualization and analysis of this complex array of data set can be quite time consuming for the physician. Current methods of both acquisition and visualization do not provide adequate information in a seamless manner to the physician to enable a productive analysis of the images. Some of the disadvantages associated with this type of analysis are related to the visualization of the injured/infracted region relatively compared to the rest of the imaging volume, including segmentation, volume analysis and visualization of the infarct throughout the cardiac phases. Also, the injured/infracted region should be characterized with temporal resolution throughout the volume sufficient for pharmacological and physical stress studies.

There remains a need for a method of evaluating tissue using 3D multi-phasic quantitative perfusion and viability imaging, in combination and separately, on whole organ systems.

### BRIEF DESCRIPTION OF THE INVENTION

In an exemplary embodiment of the invention, a method of evaluating tissue of an organ includes accessing image data and processing the image data to quantify at least one feature of interest in the tissue. The image data is derived from a computed tomography acquisition system and includes multiple phases of acquired whole organ data. Each phase is acquired within five gantry rotations of the acquisition system using an acquisition protocol.

In another exemplary embodiment of the invention, a method of evaluating tissue of a moving organ includes accessing image data and processing the image data to quantify a feature of interest in the tissue. The image data is derived from an acquisition system and includes multiple phases of acquired whole organ data. Each phase is acquired within one organ motion cycle using an acquisition protocol.

In another exemplary embodiment of the invention, a method for evaluating tissue of a moving organ includes accessing image data and processing and defining the image data. The image data is derived from an acquisition system and includes data pertaining to a plurality of phases of an imaging agent in a whole organ, following administration of the imaging agent. Each phase is acquired within one organ motion cycle using an acquisition protocol. The processing and defining the image data is used to quantify the agent distribution in the tissue, whole organ uptake of the agent, regional uptake of the agent, regional washout of the agent, regional presence of the agent, whole organ washout of the agent, clearance of the agent in the tissue over the plurality of phases, or any combination of at least one of the foregoing.

### BRIEF DESCRIPTION OF THE DRAWINGS

Referring to the exemplary drawings wherein elements are numbered alike in the several Figures:

FIG. 1 is a perspective view of an exemplary embodiment of a CT imaging system and a patient disposed for imaging according to an embodiment of the invention;

FIG. 2 is a block schematic diagram an exemplary embodiment of a CT imaging system according to an embodiment of the invention;

FIG. 3 is an exemplary embodiment of a protocol for perfusion and viability imaging according to an embodiment of the invention;

FIGS. 4 and 5 illustrate exemplary embodiments of data acquisition modes, tools and techniques within a tissue evaluation protocol according to an embodiment of the invention;

FIG. 6 illustrates exemplary embodiments of modes, tools and techniques related to contrast agent injection within a tissue evaluation protocol according to an embodiment of the invention;

FIG. 7 illustrates exemplary embodiments of modes, tools and techniques related to adjusting image contrast within a tissue evaluation protocol according to an embodiment of the invention;

FIG. 8 illustrates exemplary embodiments of modes, tools and techniques related to segmentation and sizing volumes of interest within a tissue evaluation protocol according to an embodiment of the invention;

FIG. 9 illustrates exemplary embodiments of modes, tools and techniques related to quantification of a feature of interest within a tissue evaluation protocol according to an embodiment of the invention;

FIG. 10 illustrates exemplary embodiments of modes, tools and techniques related to visualizing images within a tissue evaluation protocol according to an embodiment of the invention;

FIG. 11 illustrates exemplary embodiments of modes, tools and techniques related to reporting results within a tissue evaluation protocol according to an embodiment of the invention;

FIG. 12 illustrates exemplary embodiments of modes, tools and techniques related to fusion imaging within a tissue evaluation protocol according to an embodiment of the invention;

FIG. 13 illustrates an exemplary embodiment of a method for evaluating tissue of an organ according to an embodiment of the invention;

FIG. 14 illustrates another exemplary embodiment of a method for evaluating tissue of an organ according to an embodiment of the invention;

FIG. 15 illustrates another exemplary embodiment of a method for evaluating tissue of an organ according to an embodiment of the invention; and

FIG. 16 illustrates exemplary embodiments of modes, tools and techniques that may be used in the methods illustrated in FIGS. 13 through 15.

### DETAILED DESCRIPTION OF THE INVENTION

Disclosed herein in the exemplary embodiments are a system and methodologies that enable a streamlined workflow for 3D, multi-phasic quantitative perfusion and viability imaging on whole organ systems, with reference to a computed tomography (CT) imaging system. While an exemplary system and methodology of positioning an anatomical object relative to a CT imaging system is disclosed, it will be appreciated that such disclosure is illustrative only, and it should be understood that the method and system of the disclosed invention may readily be applied to other imaging systems, such as magnetic resonance (MR) or other scanning systems. Additionally, while the anatomical object disclosed is a heart and related myocardial tissue, it will also be appreciated that such disclosure is illustrative only, and the method and system of the disclosed invention may readily be applied to other anatomical objects, including, but not limited to a liver, brain, vasculature or kidney.

In accordance with an exemplary embodiment, a method of evaluating tissue of an organ includes deriving image data by an imaging or scanning system, such as a computed tomography (CT) system. The image data is acquired according to a specified protocol and includes multiple phases of whole organ data. Each phase is acquired within multiple gantry rotations of the imaging system, for example, five gantry rotations. The image data is accessed and processed to quantify features of interest in the tissue.

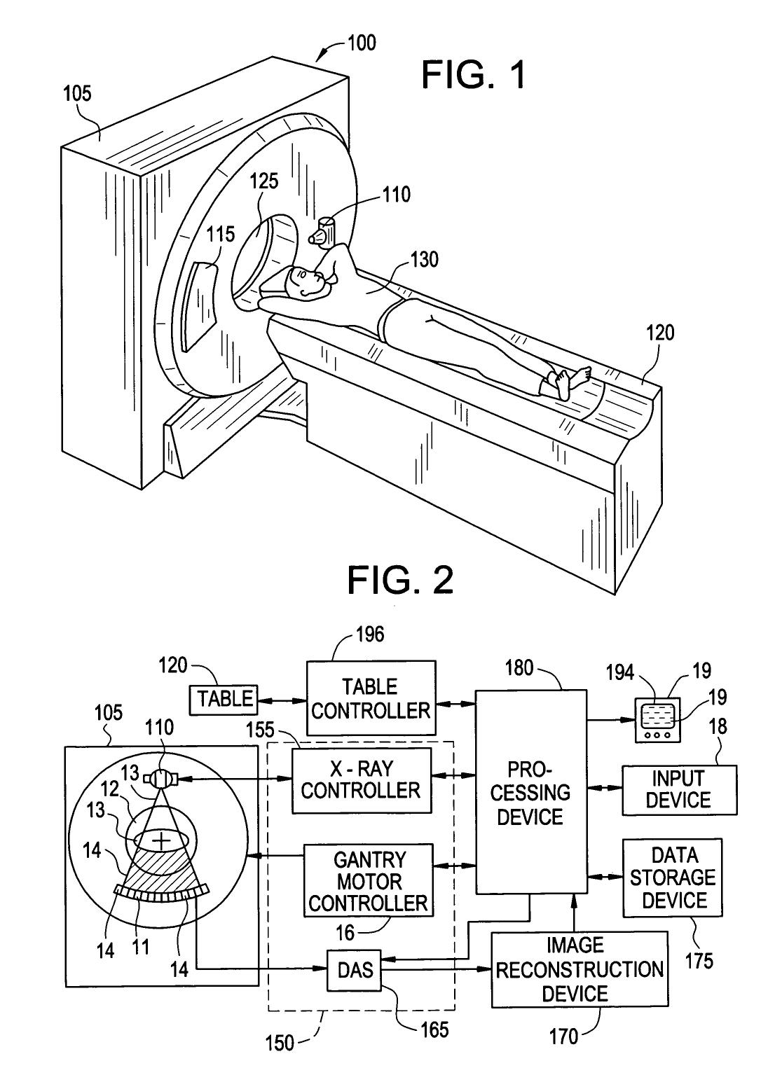

FIGS. 1 and 2 illustrate an exemplary CT imaging system 100 including a gantry 105 having an x-ray source 110, a radiation detector array 115, a patient support structure 120 and a patient cavity 125, wherein the x-ray source 110 and the radiation detector array 115 are opposingly disposed so as to be separated by the patient cavity 125. In an exemplary embodiment, a patient 130 is disposed upon the patient support structure 120, which is then disposed within the patient cavity 125. The x-ray source 110 projects an x-ray beam 135 toward the radiation detector array 115 so as to pass through the patient 130. In an exemplary embodiment, the x-ray beam 135 is collimated by a collimator (not shown) so as to lie within an X-Y plane of a Cartesian coordinate system referred to as an “imaging plane”. After passing through and becoming attenuated by the patient 130, the attenuated x-ray beam 140 is received by the radiation detector array 115. The radiation detector array 115 receives an attenuated x-ray beam 140 and produces an electrical signal responsive to the intensity of the attenuated x-ray beam 140.

The x-ray source 110 and the radiation detector array 115 are rotatingly disposed relative to the gantry 105 and the patient support structure 120, so as to allow the x-ray source 110 and the radiation detector array 115 to rotate around the patient support structure 120 when the patient support structure 120 is disposed within the patient cavity 125. X-ray projection data is obtained by rotating the x-ray source 110 and the radiation detector array 115 around the patient 130 during a scan. The x-ray source 110 and the radiation detector array 115 communicate with a control mechanism 150 associated with the CT imaging system 100. The control mechanism 150 controls the rotation and operation of the x-ray source 110 and the radiation detector array 115.

The control mechanism 150 includes an x-ray controller 155 communicating with an x-ray source 110, a gantry motor controller 160, and a data acquisition system (DAS) 165 communicating with a radiation detector array 115. The x-ray controller 155 provides power and timing signals to the x-ray source 110, the gantry motor controller 160 controls the rotational speed and angular position of the x-ray source 110, and the radiation detector array 115 and the DAS 165 receive the electrical signal data for subsequent processing.

The CT imaging system 100 also includes an image reconstruction device 170, a data storage device 175 and a processing device 180, wherein the processing device 180 communicates with the image reconstruction device 170, the gantry motor controller 160, the x-ray controller 155, the data storage device 175, an input device 185 and an output device 190. The CT imaging system 100 can also include a table controller 196 in communication with the processing device 180 and the patient support structure 120, so as to control the position of the patient support structure 120 relative to the patient cavity 125.

In accordance with an exemplary embodiment, the patient 130 is disposed on the patient support structure 120, which is then positioned by an operator via the processing device 180 so as to be disposed within the patient cavity 125. The gantry motor controller 160 is operated via processing device 180 so as to cause the x-ray source 110 and the radiation detector array 115 to rotate relative to the patient 130. The x-ray controller 155 is operated via the processing device 180 so as to cause the x-ray source 110 to emit and project a collimated x-ray beam 135 toward the radiation detector array 115 and hence toward the patient 130. The x-ray beam 135 passes through the patient 130 so as to create an attenuated x-ray beam 140, which is received by the radiation detector array 115. The radiation detector array 115 may include a plurality of detector elements 145 receiving an attenuated x-ray beam 140 and producing an electrical signal responsive to the intensity of the attenuated x-ray beam 140.

The radiation detector array 115 receives the attenuated x-ray beam 140, produces electrical signal data responsive to the intensity of the attenuated x-ray beam 140 and communicates this electrical signal data to the DAS 165. The DAS 165 then converts this electrical signal data to digital signals and communicates both the digital signals and the electrical signal data to the image reconstruction device 170, which performs high-speed image reconstruction. This information is then communicated to the processing device 180, which stores the image in the data storage device 175 and displays the digital signal as an image via output device 190. In accordance with an exemplary embodiment, the output device 190 includes a display screen 194 having a plurality of discrete pixel elements 192.

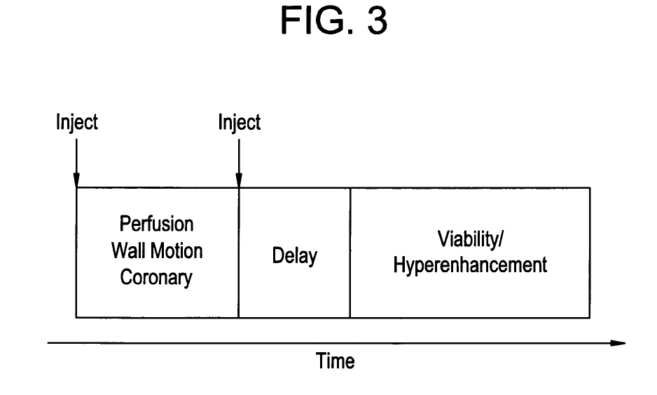

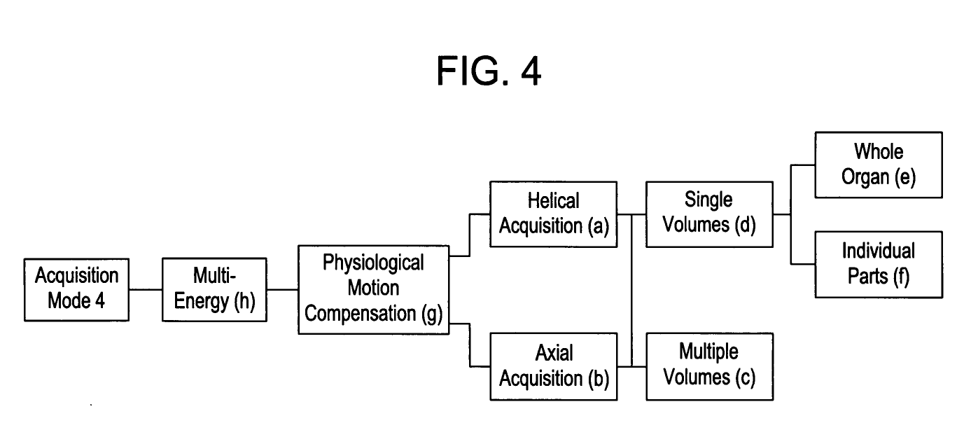

In an exemplary embodiment illustrated in FIG. 3, the protocol may include a perfusion and/or a viability (hyper-enhancement) imaging protocol. For example, the protocol may include a first injection of a contrast agent followed in a “first-pass” through the tissue, where perfusion, or flow, defects may be observed. At some time delay after the perfusion scan and a second injection of a contrast agent, a high-resolution scanning of the whole organ may be acquired. Alternatively, the protocol may also include a tissue density protocol and/or an angiographic protocol. The protocol may include various modes within the protocol. A more detailed discussion follows of the various modes, including tools and techniques for use within these modes, with respect to FIGS. 4 through 12.

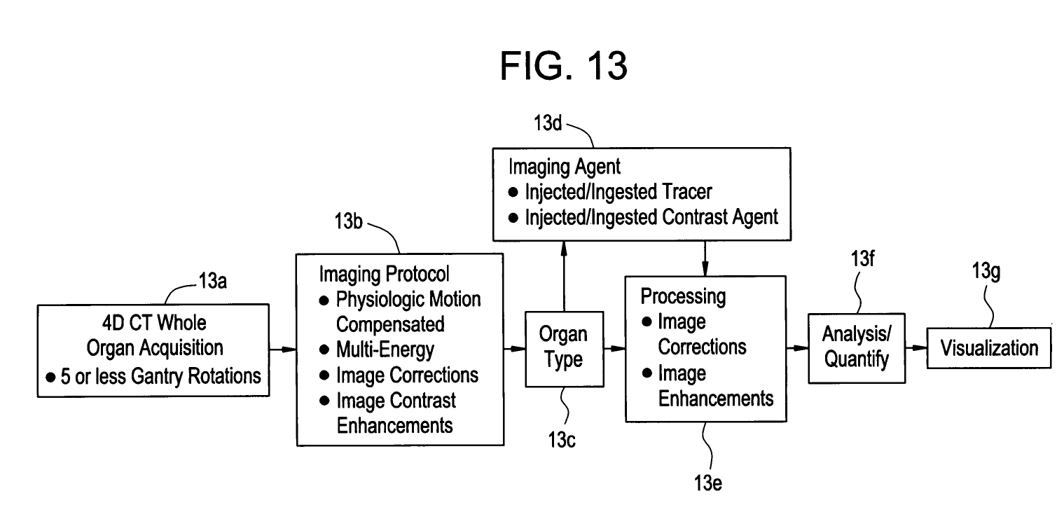

In an exemplary embodiment of a method of evaluating tissue of an organ, the image data obtained from the protocol may include data that includes multiple phases of acquired data and may include acquired whole organ data as illustrated in FIG. 13. Each phase is acquired within multiple cycles of the imaging, or acquisition, system, such as within five gantry rotations of a computed tomography acquisition system.

Phases of acquired image data may also be in response to organ motion, such as one organ motion cycle, or to injected contrast agent, such as with a contrast agent in whole organ following administration of the agent.

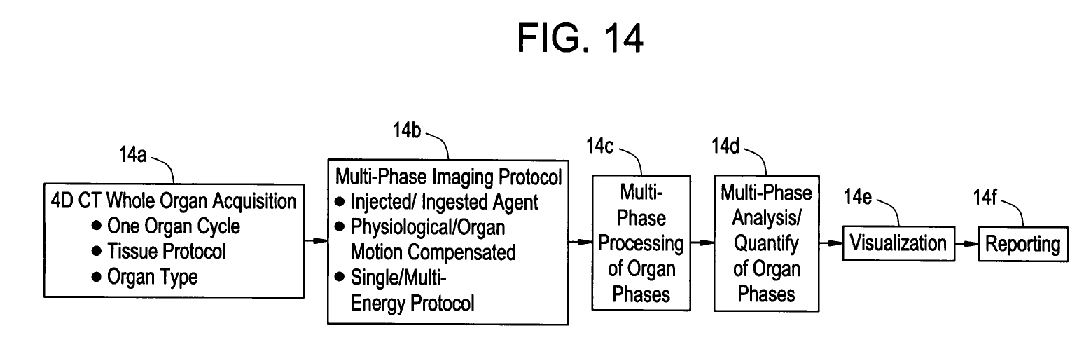

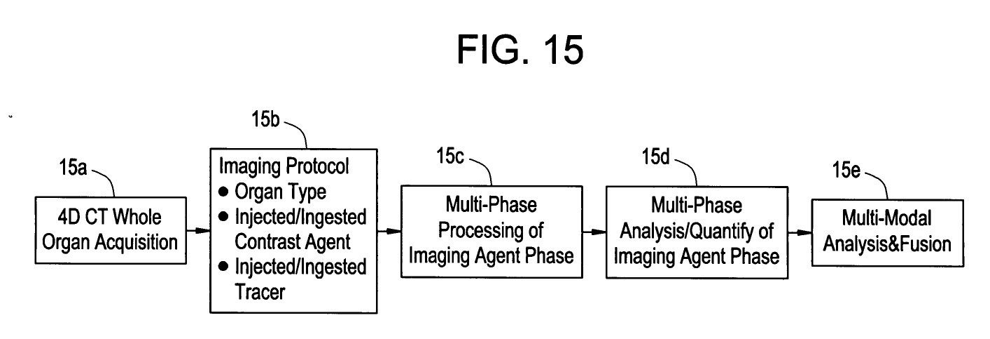

For example, in accordance with another exemplary embodiment of a method of evaluating tissue of an organ, the protocol may include deriving data from multiple phases of acquired whole organ data, each phase being acquired within one organ motion cycle as illustrated in FIG. 14. In accordance with yet another exemplary embodiment, the protocol may include deriving data from a plurality of phases of a contrast agent in a whole organ following administration of the agent, each phase being acquired within one organ motion cycle as illustrated in FIG. 15.

Once the image data is acquired, it is accessed for further processing and defining of the image data, such as to quantify features of interest in the tissue. For example, in FIG. 15, where the image data comprises data pertaining to a plurality of phases of an imaging (contrast) agent in a whole organ and each phase is acquired within one organ motion cycle, quantifiable features of interest may include the agent distribution in the tissue, whole organ uptake of the contrast agent, regional uptake of the contrast agent, regional washout of the contrast agent, regional presence of the contrast agent, whole organ washout of the contrast agent and contrast agent clearance in the tissue over the plurality of phases.

In exemplary embodiments, other features of interest may include, but are not limited to, volumes of tissue infarct, volumes of tissue injury, infarct percentage of organ, injury percentage of the organ, organ wall motion, blood flow to the tissue, permeability of tissue, extraction fraction, microvasculature density, microvasculature pattern, edema, inflammation of organ, calcifications, thermal homogeneities, pH homogeneities, stroke volume, mass volume, percent stenosis, tumor mass, excessive apotosis, high oxidative stress, neural degeneration, remodeling, thrombosis, mass shape, fiber density, cell tracking, energy absorption differences between normal and injured tissues, or any combination including at least one of the foregoing.

Referring to FIGS. 4-12, in accordance with another exemplary embodiment of a method of evaluating the tissue of an organ, the method includes modes and structures for acquiring and reconstructing 4, 5, 6, 7 image data to adjust and improve image quality, segmenting and sizing 8 volumes of interest, quantifying the volumes of interest 9, visualization 10 of multiple volumes of image data, reporting 11 both quantitative and/or qualitative parameters and fusion imaging 12 with other imaging systems or modalities.

With reference now to FIG. 4, under multi-energy acquisition 4h, physiological motion, such as from cardiac and respiratory functioning can create artifacts in images. One methodology to adjust image data 4g includes using signals created by the body, such as cardiac or respiratory, and either prospectively or retrospectively synchronizing the imaging system to the image data collected. Prospectively, the imaging system adjusts to the signal in acquiring data. Retrospectively, the data is synchronized to the signal and reconstructed to minimize artifacts. In alternative embodiments, raw acquired data and/or image data may be used to analyze the motion of the organ or anatomical part and then correct for image artifacts. Here, no signal is collected outside of the data being acquired for images. This may be done in real-time or retrospectively.

Both perfusion and hyper-enhancement viability imaging may be acquired in a helical 4a or axial 4b-4f mode as illustrated in FIG. 4. Imaging may be acquired as a single rotation (small volume) or as a helical data set. In accordance with an exemplary embodiment, imaging may include multiple axial data sets with slightly overlapped slices to compensate for physiological motion.

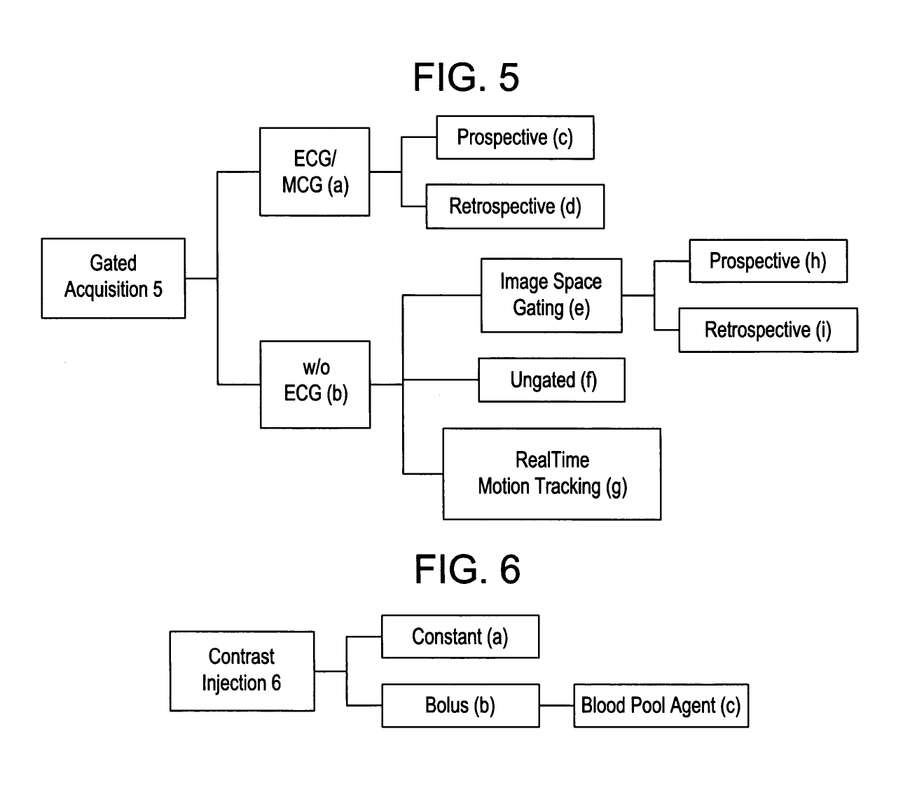

With reference now to FIG. 5, methods of gating 5 image data sets to monitor the cardiac motion include ECG 5a, which can be used to measure the depolarization of the heart or MCG (mechanical cardiac gating) 5a, which measures the closing of the valves. As illustrated in FIG. 5, these types of gating may be used in both a prospective 5c mode (acquisition changes depending on cardiac cycle) or in a retrospective mode 5d (all data is acquired and later reconstructed). Image data may also be acquired without physiological gating 5b by using a fast acquisition in an ungated mode 5f or an image space 5e method that uses changes in the images or raw data to correct for motion. This can be done prospectively 5h using the raw data or retrospectively 5i using data or images. For example, in advanced clinical applications, it may be necessary to view closed cavities, peek through an otherwise opaque lumen, or reverse the perspective of certain anatomy in 3 dimensions. Some methods which rely on the imaging of organ motion in realtime 5g by measuring changes in position of organs, such as the diaphragm, heart, great vessels or lung, provide the capability of prospectively gating the image data set.

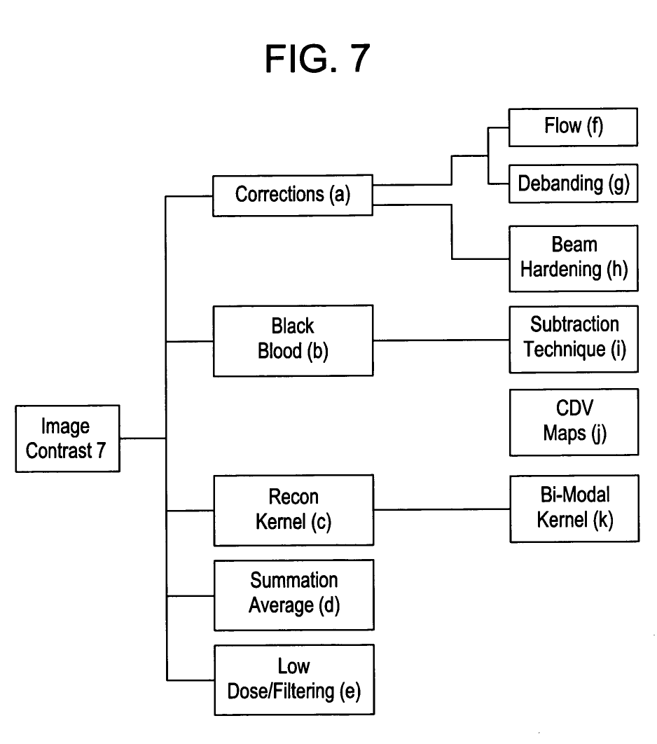

In other exemplary embodiments, acquiring and reconstructing 4, 5 data may also include pharmacological agents that affect image contrast, such as contrast agents or tracers. Referring to FIGS. 6 and 7, injection or ingestion of the agent 6 may include a bolus injection 6b, a continuous injection 6a, an iodinated contrast agent or a blood pool agent 6c.

In exemplary embodiments, myocardial viability imaging may be done with an iodinated contrast agent such as Visipaque™ manufactured by General Electric, which is short lived in the blood pool. In alternative embodiments, a longer lasting blood pool agent may be used, which remains in the blood for an extended period of time. Here, it is possible to acquire steady-state images of multiple vascular regions over an hour or more with a single injection, rather than chasing a conventional contrast bolus for a shorter period of time, for example, 30 seconds. Advantageously, this may allow one injection for the perfusion and viability study. As a further advantage, longer lasting blood pool agents do not overestimate the size of the infarct as much as other contrast agents such as gadolinium diethylenetriaminepentaacetic acid (Gd-DTPA) in MRI.

With reference now to FIG. 7, perfusion defects show up as a hypo-enhancement and non-viable tissue appears as hyper-enhancements within the viability image. Both of these effects can be small in relation to signals surrounding the perfusion defects and non-viable tissue from the blood pool and normal myocardium. Within the viability images, the blood pool is generally bright from the contrast agent and can mask the effect of the non-viable tissue. To improve image quality and the image contrast perfusion defects and non-viable tissue, corrections 7a, summation of the images 7d, black blood imaging 7b, reconstruction kernels 7c and low dose filtering 7e may be included as a tool in the method of evaluating tissue as illustrated in FIG. 7.

Corrections 7a includes flow corrections 7f, debanding 7g or beam hardening 7h. Summation 7d may include summing the images from the same part of the cardiac cycle. Adding the images increases the contrast between the low flow regions and the viable myocardium. Black blood imaging 7b employs a subtraction technique 7i between contrast images from the perfusion data set and the hyper-enhancement data set to make the blood pool appear dark to increase contrast. Black blood imaging 7b may also include current density vector (CDV) maps 7j. In a bi-modal image reconstruction kernel 7k, a reconstruction kernel or view weighting may be applied to moderate contrast between the myocardium and the blood pool. The lower frequencies define the contrast between the myocardium and the blood pool and the higher frequencies increase the spatial resolution and help define the edges of the infarct and myocardial wall. Low dose filtering 7e including dose reduction protocols may also be used as they are an valuable consideration for embodiments including imaging where two different data set will be acquired.

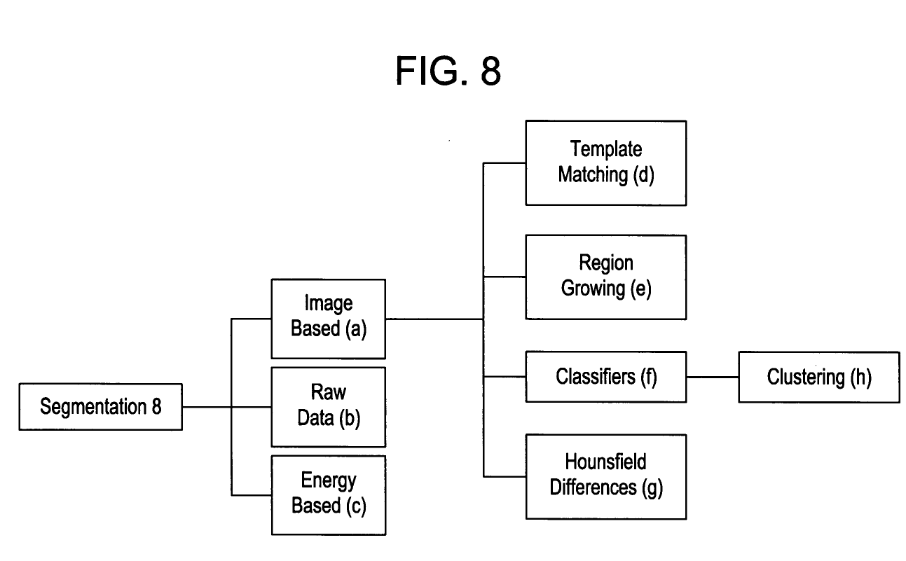

With reference now to FIG. 8, the method of evaluating tissue may also include automated or semi-automated segmentation and sizing 8 of volumes of interest, such as cardiac infarcts. Segmentation and sizing 8 of cardiac infarcts play an important clinical role in patient care for the purpose of detection, diagnosis, planning for care, treatment, and follow-up. Cardiac infarcts may only be visible at different phases of the cardiac cycle. In exemplary embodiments, segmentation may be image based 8a or based on raw image data 8b. Segmentation may also be energy-based 8c, such as when the acquisition system is configured for multi-energy acquisition. For example, a multi-energy CT system may be configured to allow simultaneous capture of several sets of sinogram data, each having a unique x-ray energy distribution.

An infarct may be segmented 8 using template imaging 8d, classifiers 8f, Hounsfield units 8g or region growing 8e within the volume of the ischemic region. Where image data includes multiple phases of acquired data, processing of the image data may include segmentation of tissue characteristics of at least one phase. A statistical classification technique, also known as clustering 8h, may be used to determine where certain populations of image data fall within different groups. Quantitative comparisons may be made of multiple characteristics. These characteristics include, but are not limited to, signal intensity, Hounsfield units and spatial location in the image. Clustering 8h may be done in two or three dimensions. In exemplary embodiments, clustering 8h may be done temporally to essentially add a fourth dimension to the image data.

For example, in combination with wall motion data, which may be acquired with initial perfusion data, a fraction of the wall that the infarct is involved in may be quantified. The segmentation 8 of the infarct may be computed through various cardiac phases to obtain quantitative information. The wall thickness and percent of transmurality may also be obtained along the complete timeline of the cardiac phases. In other exemplary embodiments, where the image data comprises data pertaining to a plurality of phases of a contrast agent in a whole organ and each phase is acquired within one organ motion cycle, processing the image data includes segmentation of contrast distribution within the organ and/or segmentation of contrast clearance throughout the organ.

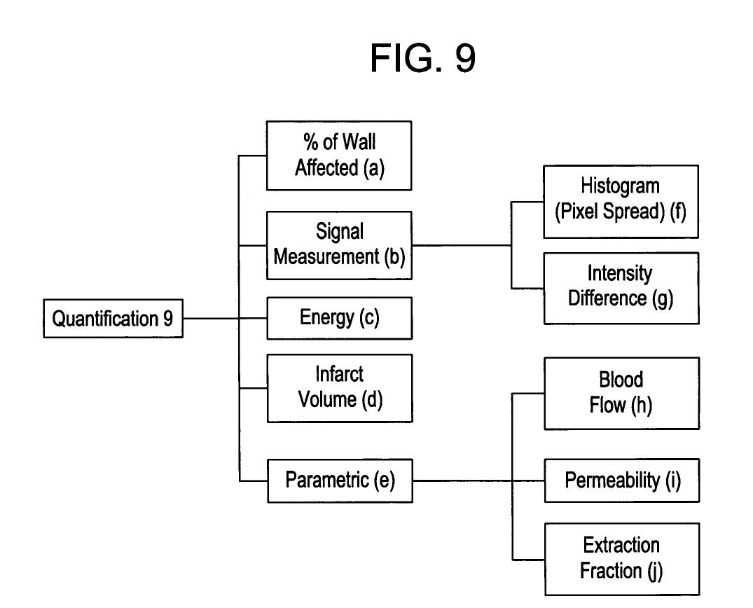

With reference now to FIG. 9, in exemplary embodiments, both perfusion and viability imaging may be quantified 9 with multi-phasic CT. In quantifying features of interest within the organ or tissue, parameters 9e such as blood flow 9h, blood volume, extraction fraction 9j and permeability 9i may be assessed from both perfusion and viability data. These parameters may vary throughout the cardiac cycle and therefore may need to be assessed continually. In other exemplary embodiments, parameters such as the percent area of the wall 9a, the intensity differential 9g and histogram of pixels 9f within the infracted volume from signal measurement 9b, an amount of energy 9c and total volume of the infarct 9d may be evaluated for disparity throughout the cardiac phases.

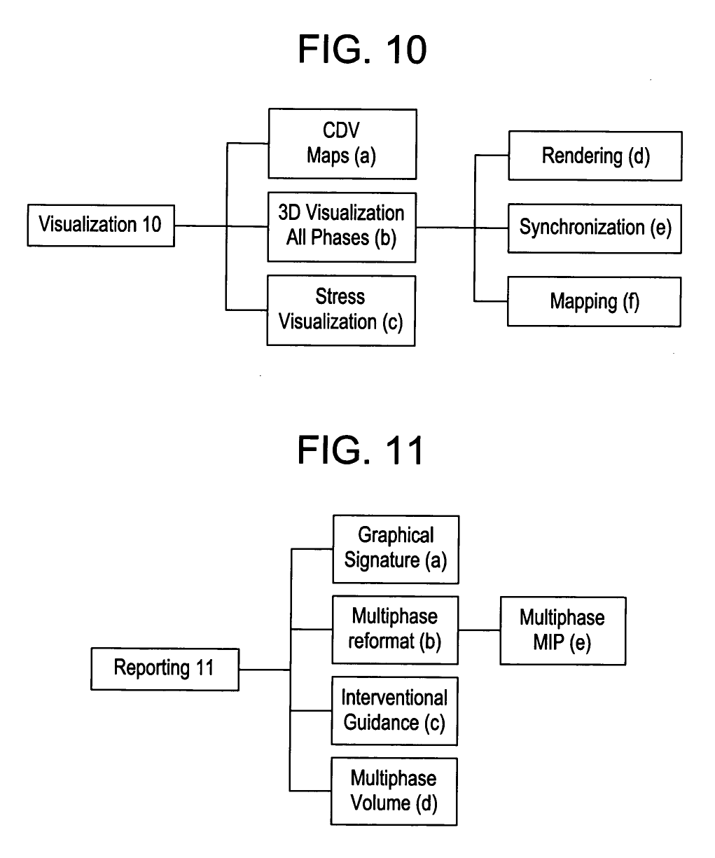

With reference now to FIG. 10, for viability imaging, whole heart data may be collected as a helical data set or as a series of axial images combined to make a complete volume. Viability imaging may use only one volume collected and reconstructed at one time point, such as at 70-75% of the cardiac cycle. A complete analysis of the heart over all cardiac phases may also be performed. Modes and techniques are needed for care agents to visualize multiple volumes 10 of image data. For example, care agents may use manual/automated visual synchronization 10e, mapping 10f and rendering 10d to view different 3D and 4D image sets 10b of the anatomical part. Some of the automated synchronized 10e visualization may require the registration of different 3D or 4D datasets. The infarct may be able to be seen completely within the 3D volume and also as a transparent volume using volume rendering 10d, allowing the surgeon to plan according to anatomical landmarks as well as relative distances. Multiphase distribution maps, such as CDV maps 10a of the contrast agent may also be used to increase the conspicuity of an ischemic region.

In another exemplary embodiment, a “stress study” may be performed where the patient's heart rate is affected by either medication or exercise. Here, visualization 10, 10c is required not only at different locations in the organ, or heart, but also at different heart rates. This visualization 10c may include multiple, time synchronized windows (to the R-peak) of each region of infarct comparatively along a timeframe of increased heart rate.

In alternative embodiments, side-by-side comparisons may also be performed between the perfusion imaging and the myocardial viability imaging. This comparison may include, but is not limited to, synchronization 10e of the visualization of infarcts over different phases or temporal 3D images. In addition to automated methods of synchronization 10e, the infarct may be viewed in 3D at various cardiac phases. For example, where the image data comprises data pertaining to a plurality of phases of a contrast agent in a whole organ and each phase is acquired within one organ motion cycle, a viability analysis or viability protocol includes visualization of multi-phase parameters, such as illustrated in FIG. 14.

With reference now to FIG. 11, since CT is able to accurately quantify the effect from the contrast compared to other modalities, such as MRI, a structure of reporting 11 the results of the imaging and evaluation may contain additional information from the acquired image data. The additional information may include, but is not limited to, 3D multi-phase quantitative maps of parametric values, for example, blood flow 9h, blood volume, permeability 9i and extraction fraction 9j, multi-phasic graphical signatures 11a at various voxels, maximum intensity profiles (MIP) 11e via multiphase reformatting 11b, interventional guidance 11c and volumetric summaries 11d of the ischemic volume in a multi-phasic display in a physician report.

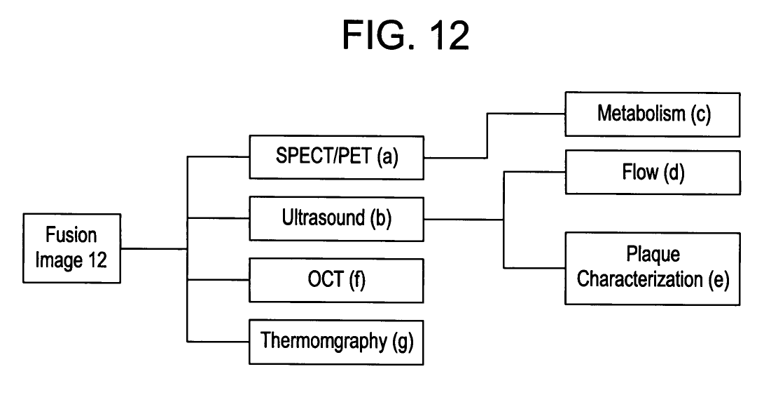

With reference now to FIG. 12, in exemplary embodiments, the acquisition system, such as a multi-phasic CT imaging system, may be used in combination with other modalities to provide addition information to the care agent. This use in combination with a second modality may also be known as a fusion image mode 12. The acquisition system and the other modalities may include, but are not limited to, positron emission tomography (PET) 12a, single photon emission computed tomography (SPECT) 12a, ultrasound 12b, magnetic resonance imaging (MRI), optical coherence tomography (OCT), or thermography, intravascular ultrasound (WUS). For example, CT provides some functional information about the viability of the myocytes, but PET and SPECT 12a may provide additional 4D information about metabolism 12c and perfusion within the microvasculature. Ultrasound 12b may provide information relative to flow 12d and plaque characterization 12e. Additionally, the other modalities such as IVUS, Thermomgraphy 12g, or OCT (Optical Coherence Tomography) 12f can measure high oxidative stress, excessive apoptosis, temperature inhomogenities or pH inhomogenities. may provide information concerning the cause of the ischemia.

Referring to FIGS. 13 through 15, these aforementioned various modes and structures may be combined within exemplary embodiments of a method of evaluating tissue.

For example, in accordance with another exemplary embodiment of a method of evaluating tissue of an organ illustrated in FIG. 13, the method includes accessing image data that is derived from a computed tomography acquisition system 13a. The image data includes multiple phases of acquired whole organ data 13a. Each phase is acquired within five gantry rotations 13a of the acquisition system using an acquisition protocol 13b. The imaging protocol 13b as illustrated includes physiological motion compensation 4g, multi-energy acquisition 8c and image corrections 7, 7a and contrast enhancements 6. Based on organ type 13c, an imaging agent 6, 13d may be used. The image data is processed 13e to quantify 13f a feature of interest in the tissue, whereby visualization 10, 13g may be used to further evaluate the tissue.

In accordance with another exemplary embodiment of a method of evaluating tissue of an organ illustrated in FIG. 14, the method includes accessing image data that is derived from an acquisition system 14a. The image data includes multiple phases 14b of acquired whole organ data. Each phase is acquired within one organ motion cycle 14a using an acquisition protocol 14a based on the organ type 14a. The imaging protocol 14b as illustrated may include physiological motion compensation 4g, multi-energy acquisition 8c and image injected or ingested image contrast agent 6. The image data is processed 14c to quantify 14d a feature of interest in the tissue, whereby visualization 10, 14e may be used to further evaluate the tissue. The results of the imaging and evaluation may be reported 11, 14f and contain additional information from the acquired image data as discussed above.

In accordance with another exemplary embodiment of a method of evaluating tissue of an organ illustrated in FIG. 15, the method includes accessing image data that is derived from an acquisition system 15a. The image data includes data pertaining to a plurality of phases of a contrast agent in a whole organ 15a following administration of a contrast agent 15b. Each phase is acquired within one organ motion cycle using an acquisition protocol 15b. The image data is processed and defined 15c to quantify 15d, 9 contrast distribution in the tissue, whole organ uptake of contrast agent, regional uptake of contrast agent, regional washout of contrast agent, whole organ washout of contrast agent, contrast clearance in the tissue over the plurality of phases. The image data may be further analyzed and by interacting with another acquisition system or modality in a fusion mode 12, 15e.

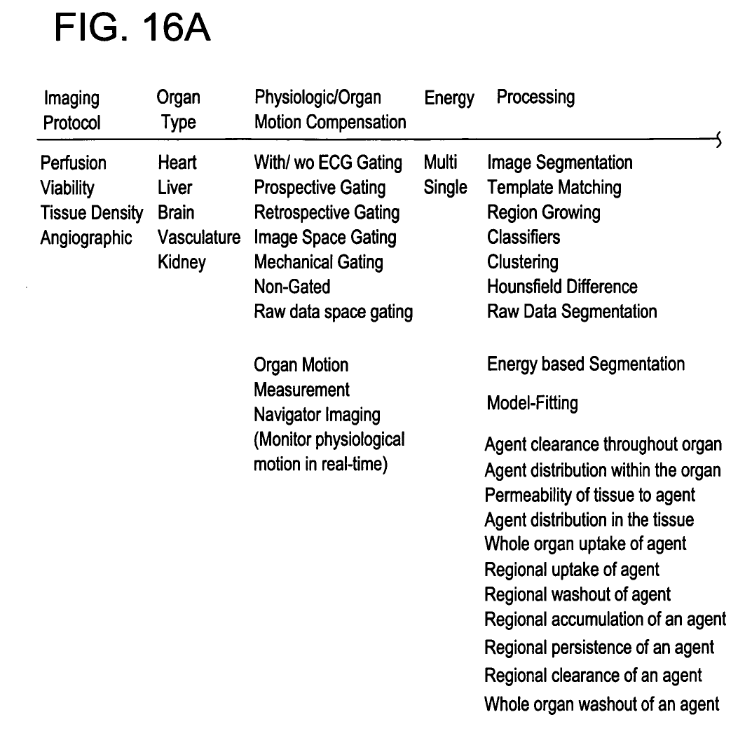

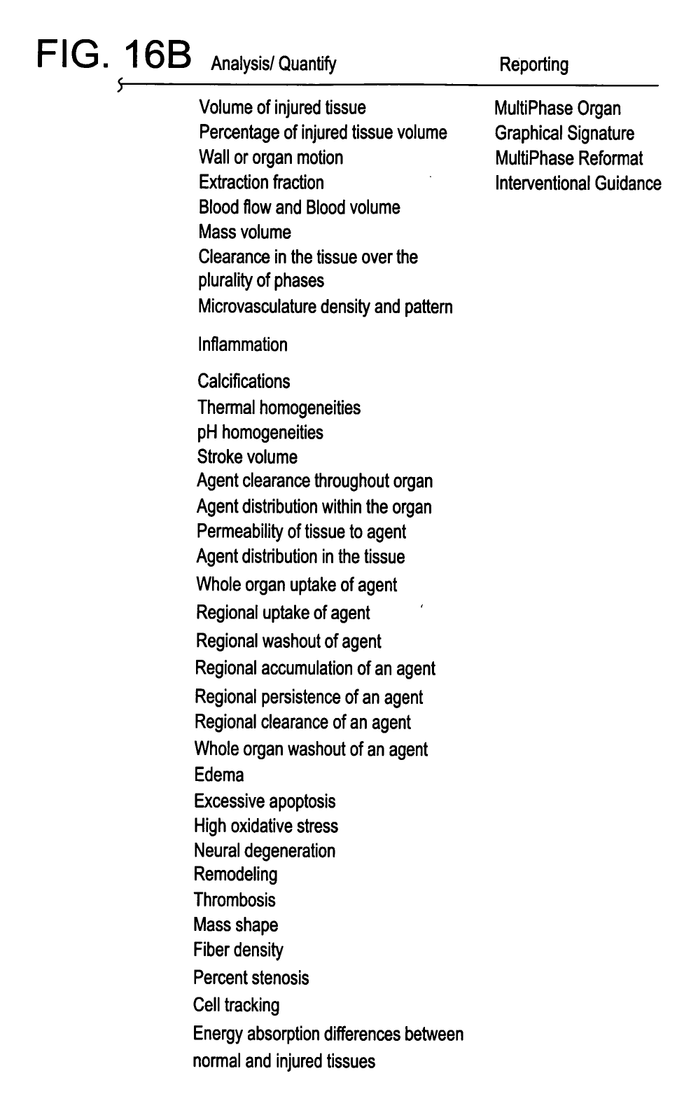

Various modes and structures that may be used in the exemplary embodiments illustrated in FIGS. 13-15 are detailed in FIG. 16.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed for carrying out this invention, but that the invention will include all embodiments falling within the scope of the appended claims.

1. A method of evaluating tissue of an organ, the method comprising:
accessing image data derived from a computed tomography acquisition system wherein the image data comprises multiple phases of acquired whole organ data wherein each phase is acquired within five gantry rotations of the acquisition system using an acquisition protocol; and
processing the image data to quantify at least one feature of interest in the tissue.

2. The method of claim 1 wherein the phases of acquired whole organ data are in response to organ motion, an injected contrast agent, an injected tracer, an ingested tracer, or any combination comprising at least one of the foregoing.

3. The method of claim 1 wherein the protocol comprises a tissue perfusion protocol, a tissue viability protocol, a tissue density protocol, an angiographic protocol, or any combination comprising at least one of the foregoing.

4. The method of claim 1 wherein the feature of interest comprises volumes of tissue infarct, infarct percentage of organ, organ wall motion, blood flow to the tissue, permeability of tissue, extraction fraction, microvasculature density, microvasculature pattern, edema, inflammation of organ, calcifications, thermal homogeneities, pH homogeneities, stroke volume, mass volume, percent stenosis, tumor mass, agent distribution within the organ, agent clearance throughout the organ, agent distribution in the tissue, whole organ uptake of agent, regional uptake of agent, regional washout of agent, regional accumulation of an agent, regional persistence of an agent, regional clearance of an agent, whole organ washout of an agent, clearance in the tissue over the plurality of phases, excessive apotosis, high oxidative stress, neural degeneration, remodeling, thrombosis, mass shape, fiber density, cell tracking, energy absorption differences between normal and injured tissues, or any combination comprising at least one of the foregoing.

5. The method of claim 1 wherein the organ is a heart, liver, brain, vasculature, or kidney.

6. The method of claim 1 further comprising visualization of the feature of interest.

7. The method of claim 1 wherein the acquisition system is configured for multi-energy acquisition.

8. The method of claim 1 wherein the processing the image data comprises defining and processing tissue characteristics of at least one phase.

9. A method of evaluating tissue of a moving organ, the method comprising:
accessing image data derived from an acquisition system wherein the image data comprises multiple phases of acquired whole organ data wherein each phase is acquired within one organ motion cycle using an acquisition protocol; and
processing the image data to quantify a feature of interest in the tissue.

10. The method of claim 9 wherein the phases of acquired whole organ data are in response to organ motion, an injected imaging agent, or both.

11. The method of claim 9 wherein the protocol comprises a tissue perfusion protocol, a tissue viability protocol, or both.

12. The method of claim 9 wherein the feature of interest comprises volumes of tissue infarct, organ wall motion, blood flow to the tissue, permeability of tissue, extraction fraction, microvasculature density, microvasculature pattern, edema, inflammation of organ, calcifications, thermal homogeneities, pH homogeneities, stroke volume, mass volume, tumor mass, percent stenosis, agent distribution within the organ, agent clearance throughout the organ, agent distribution in the tissue, whole organ uptake of agent, regional uptake of agent, regional washout of agent, regional accumulation of an agent, regional persistence of an agent, regional clearance of an agent, whole organ washout of an agent, clearance in the tissue over the plurality of phases, excessive apoptosis, high oxidative stress, neural degeneration, remodeling, thrombosis, mass shape, fiber density, cell tracking, energy absorption differences between normal and injured tissues, or any combination comprising at least one of the foregoing.

13. The method of claim 9 further comprising visualizing the feature of interest.

14. The method of claim 9 further comprising reporting results of the quantified feature of interest.

15. A method for evaluating tissue of a moving organ, the method comprising:
accessing image data derived from an acquisition system wherein the image data comprises data pertaining to a plurality of phases of an imaging agent in a whole organ following administration of the imaging agent wherein each phase is acquired within one organ motion cycle using an acquisition protocol; and
processing and defining the image data to quantify the agent distribution in the tissue, whole organ uptake of the agent, regional uptake of the agent, regional washout of the agent, regional presence of the agent, whole organ washout of the agent, clearance of the agent in the tissue over the plurality of phases, or any combination comprising at least one of the foregoing.

16. The method of claim 15 wherein the processing and defining the image data comprises processing and defining of the agent distribution within the organ, processing and defining clearance of the agent throughout the organ, processing and defining of the agent distribution in the tissue, processing and defining of whole organ uptake of the agent, processing and defining of regional uptake of the agent, processing and defining of regional washout of the agent, processing and defining of regional accumulation of the agent, processing and defining of regional persistence of the agent, processing and defining of regional clearance of the agent, processing and defining of whole organ washout of the agent, processing and defining of clearance of the agent in the tissue over the plurality of phases, or any combination comprising at least one of the foregoing.

17. The method of claim 15 wherein the protocol comprises a tissue perfusion analysis, a tissue viability analysis, or both.

18. The method of claim 17 wherein tissue viability analysis includes visualization of multi-phase parameters.

19. The method of claim 15, wherein the acquisition system comprises computed tomography (CT), positron emission tomography (PET), single photon emission computed tomography (SPECT), magnetic resonance imaging (MRI), ultrasound, optical coherence tomography (OCT), thermography, intravascular ultrasound (IVUS), or any combination comprising at least one of the foregoing.

20. The method of claim 15, further comprising
interacting the acquisition system with a second acquisition system in a fusion image mode, the second acquisition system comprising computed tomography (CT), positron emission tomography (PET), single photon emission computed tomography (SPECT), magnetic resonance imaging (MRI), ultrasound, optical coherence tomography (OCT), thermography, intravascular ultrasound (IVUS), or any combination including at least one of the foregoing.
